(19) United States
(12) Patent Application Publication
(10) Pub. No.: US 2025/0134545 A1
(43) Pub. Date: May 1, 2025

(54) SURGICAL CUTTING INSTRUMENT

(71) Applicants: Paul D. PATERSON, Vero Beach, FL (US); Michele PRESSACCO, Martignacco (IT)

(72) Inventors: Paul D. PATERSON, Vero Beach, FL (US); Michele PRESSACCO, Martignacco (IT)

(21) Appl. No.: 18/920,429

(22) Filed: Oct. 18, 2024

Related U.S. Application Data
(60) Provisional application No. 63/593,276, filed on Oct. 26, 2023.

(51) Int. Cl. A61B 17/32; A61B 17/00

(52) U.S. Cl. CPC A61B 17/320036; A61B 2017/00477

(57) ABSTRACT

A surgical cutting instrument for minimally invasive surgery is provided having a gripping element, an actuating element operatable by a surgeon at the gripping element, a hollow stem mounted as an extension of the gripping element along a longitudinal axis of the instrument, a blade element slidable along the longitudinal axis of the stem, a tie rod housed in the hollow stem and having a proximal end coupled to the actuating element and a distal end coupled to the blade element. A motion of the actuating element translates the blade element along the longitudinal axis of the hollow stem and moves the blade element from a distal inactive position, with the blade element hidden within the hollow stem, to a proximal active position, with the blade extracted from the hollow stem and vice versa.

### CROSS-REFERENCE TO RELATED APPLICATIONS

This application claims the benefit of priority from U.S. Provisional Application Ser. No. 63/593,276, filed Oct. 26, 2023, the contents of which are incorporated herein by reference.

### FIELD OF APPLICATION

The present invention relates to a surgical cutting instrument for minimally invasive surgery.

In general, the present invention finds application in the surgical field, in particular for the interventions related to carpal tunnel, and even more specifically, but not limitedly, to interventions for the release of the carpal tunnel by cutting the transverse ligament by means of a minimally invasive ultrasound-guided technique.

### BACKGROUND OF THE INVENTION

It is known from statistical data that 3.7% of the general population suffers from disorders related to carpal tunnel syndrome and even 7% of unskilled workers.

In the United States the incidence of carpal tunnel syndrome (CTS) is 12,000,000 people. Among those 2.7 million are indicated to have surgical treatment but only 500,000 people are treated surgically each year through a procedure known as the carpal tunnel release (CTR).

During the carpal tunnel release procedure the transverse carpal ligament is cut in order to reduce the compression of the median nerve and the pressure in the carpal tunnel region.

50% of carpal tunnel release procedures are made through a so-called “open” surgical technique, the remaining 50% through an endoscopic technique.

Although these surgical techniques report good clinical results in at least 97% of cases, many people (2.1 million) prefer not to undergo any treatment in order to avoid a long period of convalescence and a long clinical course which requires several specialist visits within 6-9 months (typically 5-6 visits).

Minimally invasive ultrasound-guided techniques have recently been developed in order to overcome the limitations of conventional techniques and therefore reduce recovery times, reduce pain, avoid stitches, improve patient satisfaction, and therefore reduce treatment costs.

In implementing these techniques, it is important that the cutting blade is covered both when the surgeon inserts the instrument into the carpal canal and when he extracts it, in order to avoid accidental damage to the tissues of this anatomical site.

The carpal canal is a cavity located at the level of the wrist.

In the anatomical position there are the carpal bones posteriorly and the transverse carpal ligament anteriorly.

In addition to the median nerve, the veins and tendons of the flexor muscles of the fingers pass through the tunnel thus formed.

It is therefore well understood that in such a small space it is of fundamental importance to extract the blade only when there is absolute certainty that only the tissue of interest will be cut, i.e. the transverse carpal ligament.

It is also preferable for the blade to be covered when the instrument is not being used.

US 2017/0042565 A1, U.S. Pat. No. 10,357,272 B2 and EP 3193748 B1 patent applications present a first solution in this regard.

In particular, the cutting element comes out of the seat due to the elastic deformation of the element itself and, even more specifically, the extraction of the blade takes place through defined paths thanks to the elastic deformation of the stem on which it is mounted.

This solution certainly has the advantage of being simple and thus very cheap.

However, there is also the obvious disadvantage of having a cutting instrument that is not particularly rigid.

Vice versa, it would be difficult to elastically deform the blade with just the push of the fingers of the surgeon who is operating the instrument.

This represents a limitation because on the one hand the deformability of the instrument prevents the surgeon from having a haptic perception in the moment when he operates, on the other hand in the case of tissues, such as for instance particularly tenacious ligaments, it is difficult to make the cut safely.

Patent application and US 2023/200833 A1 presents a different solution. Here, the blade is extracted through rotation. The rotation of the blade can be achieved in two different ways. In a first embodiment, a rail with two inclined segments and a horizontal one imparts the rotation, through a pin which slides therein. When the pin slides through the inclined rail the blade rotate upwards or downwards, while when in the horizontal segment it remains extracted for performing the resection. In a second embodiment the rotation is induced by a cam mechanism, where the blade engages different cams that induce the rotations (upwards and downwards).

The drawback of these solutions is that, even though they have a rigid layout hence providing the required haptic feedback, given their mechanical complexity they have a relatively high production cost, especially in view of the disposable nature of the device.

Another drawback, which is common to all prior art solutions, is the lack of efficiency in the procedure in case of strong ligaments. It is known that particularly tenacious ligaments, such as the ligaments of big male patients, may require two consecutive passages of the blade to be resected. Since the prior art instruments feature a blade with only a single cutting edge, the surgeon has to move the instrument back and forth at least two times. Providing a cutting edge on both sides of the blade creates efficiency and avoids possible soft tissue entanglement returning the blade in the cutting position. This entanglement could result in patient injury.

For this reason, the technical problem underlying the present invention is to provide an instrument able to overcome the above limits of the prior art.

An object of the invention is to make a rigid instrument in order to give the surgeon a haptic perception, while maintaining the instrument cost effective.

Still an object of the present invention is to provide an instrument able to facilitate the detachment of the soft tissues of the palmar arch, in particular prior to carrying out the cut of the transverse carpal ligament.

A further object is to provide an instrument which allows having reference markers in the instrument that are distinguishable from the images obtained by ultrasound examination.

A further object is to provide an instrument which allows cutting even particularly tenacious ligaments through a single back-and-forth movement of the blade.

Finally, an object of the invention is to provide a solution that is easy to use daily for a surgeon.

### SUMMARY OF THE INVENTION

The solution idea underlying the present invention is to provide an instrument which maintains the advantages deriving from a hidden solution, but which does not require an elastic deformation for the extraction and retraction phases and which at the same time does not employ relatively complex inner guides or cams.

The above technical problem is solved by a surgical cutting instrument for minimally invasive surgery comprising:
a gripping element;
an actuating element operatable by a surgeon at said gripping element;
a hollow stem mounted as an extension of said gripping element along a longitudinal axis of the instrument;
a blade element slidable along the longitudinal axis of the stem;
a tie rod housed in the hollow stem and having a proximal end coupled to the actuating element and a distal end coupled to the blade element, so that a motion of the actuating element translates the blade element along the longitudinal axis of the hollow stem;
wherein said hollow stem comprises: a distal covering tip transversally wider or with the same width of the blade and fully covering the blade; and an open portion proximal to said covering tip, transversally narrower than the blade and comprising an upper slit;
wherein the motion of the actuating element moves the blade from a distal inactive position, wherein the blade is hidden within the covering tip, to a proximal active position, wherein the blade protrudes from the upper slit.

In this embodiment, the blade does not rotate but only translates along the hollow stem as the actuating element is manipulated by the surgeon, thereby pulling the tie rod.

It should be noted that the covering tip is not necessarily integral with the rest of the hollow stem; it can be a separate piece which is welded or otherwise attached to the rest of the hollow stem.

Advantageously, the present solution allows having an instrument with a hidden blade, adapted to perform the minimally invasive surgery, but having such a rigidity as to allow the surgeon to maintain the perfect haptic perception for the execution of the operation.

Moreover, advantageously, the simple design implying mere translation of the blade by a tie rod greatly reduces the production cost with respect to prior art devices.

The actuating element can be conveniently designed as a slider which slides along the longitudinal axis. Alternatively, the actuating element can be a lever which is hinged to the proximal end of the tie-rod. In both cases, the surgeon can proceed with one hand and in a simple and intuitive way to pass the cutting instrument from an inactive phase to an active phase and vice versa.

Furthermore preferably, the actuating element is housed and sliding at a seat formed in the gripping element.

Moreover, the tip of the cutting instrument preferably comprises a nose designed to facilitate the insertion of the instrument and a crest that acts has a freer for the detachment of the soft tissues. The protuberance is also adapted to provide a housing to the blade when it is in the rest position namely the more distal end. The surgeon, once he/she has performed the cut, pushes back the blade into the tip before the extraction of the instrument from the incision.

Advantageously, the blade element can have a triangular shape with two cutting edges so that is capable to cut on both sliding directions while the planar base of the blade acts as a guide and stabilizing element during translation. Alternatively, the guidance of the blade element can be achieved by one or two blade pins sliding within an internal groove of the hollow stem. The same pin or pins can be used to couple the blade element with the tie rod. This makes it possible to make the hollow stem tubular without having to machine a flat portion for guiding the planar base. A further option is that of welding the blade element to the tie rod which is then guided by the inner circumference of the tubular hollow stem.

As previously mentioned, it is not unusual that in big male patients the resection of the transverse ligament requires two consecutive passages of the blade. Thanks to the two cutting edges the surgeon has the advantage that he/she can perform the second passage while pushing back the blade into the housing, thus saving time and increasing efficiency of the procedure.

Preferably, the gripping element comprises a left half shell and a right half shell coupled to each other so as to enclose a portion of the hollow stem, this solution being optimal for production and assembly.

Preferably, the cutting instrument has references, for instance vertical reference recesses, which can be detected by ultrasounds to identify a position of the instrument with respect to the surrounding tissues. This facilitates the surgeon in the correct identification of the position of the instrument with respect to the surrounding tissues.

The technical problem outlined above is also solved by a surgical cutting instrument for minimally invasive surgery comprising:
a gripping element;
an actuating element operatable by a surgeon at said gripping element;
a hollow stem mounted as an extension of said gripping element along a longitudinal axis of the instrument and comprising an upper slit;
a blade element slidable along the longitudinal axis of the stem;
a tie rod extending along the longitudinal axis, housed in the hollow stem and having a proximal end coupled to the actuating element and a distal end rotatably coupled at a first hinging point of the blade element, so that a motion of the actuating element translates the blade element along the longitudinal axis of the hollow stem or rotates it when a second hinging point on the blade element is kept fixed;
an auxiliary element guided along the longitudinal axis within the hollow stem and rotatably coupled at the second hinging point of the blade element, the second hinging point being spaced apart from the first hinging point;
a retaining device that prevents translation from an initial position of the auxiliary element along the hollow stem in a distal-to-proximal direction until an unlocking pulling force is transmitted to the auxiliary element, and that locks the auxiliary rod in the initial position when pulled in a proximal-to-distal direction;
wherein the relative motion between the tie rod and the auxiliary element achieved when the tie rod is displaced by the actuating element and the auxiliary element is locked by the retaining device imparts a rotating motion to the blade from an inactive position, wherein the blade is hidden within the hollow stem, to an active position, wherein the blade protrudes from the upper slit; and vice versa.

In other words, the blade is not solidly attached to the tie rod but assembled with one pin, whereas a second pin is attached to the auxiliary element, thereby allowing the rotation of the blade about an axis and the translation thereof. The blade is housed inside the hollow stem and is extracted from the rest position by means of the rotation.

The auxiliary element is preferably a rod having a proximal end selectively retained by the retaining device and a distal end rotatably coupled at the second hinging point of the blade element. Preferably, the rod can be inserted in a traversing hole integral with the actuating element for further guidance.

Preferably, the retaining device is on the gripping element, most preferably at the actuating element. Therefore, it is not constrained to the necessarily slender design of the hollow stem to be inserted in the patient body.

Preferably, the retaining device comprises a compliant retaining element selectively preventing the translation of the auxiliary element in the distal-to-proximal direction. The compliant retaining element can be an elastic tooth acting on a pin connected to the auxiliary element. If the auxiliary element is a rod, the pin can be coaxial to the free proximal end of said rod.

Preferably, the retaining device comprises an abutment preventing the translation of the auxiliary element in the proximal-to-distal direction.

Preferably, in said second embodiment with a rotating blade element the blade element is C-shaped and has only a single cutting edge. Alternatively, as above, a blade element with two cutting edges can be advantageously employed, for instance a triangle-shaped blade element.

Preferably, as in the first embodiment the actuating element can be a slider translatable along the longitudinal axis of the stem. The slider can be partially housed and guided within the gripping element.

Preferably, the surgical cutting instrument further comprises a tip coupled at the blade element and comprising a protuberance for the detachment of the soft tissues. The protuberance is further adapted to provide a spatial positioning feedback of the surgical cutting instrument in the working condition.

Advantageously, the present solution allows detaching and removing the soft tissues prior to carrying out the cutting of the ligament thanks to the freer that is designed on top of the protuberance, thus increasing the safety of the intervention and also allows the surgeon to know where the instrument is when inserted thanks to the combination of the crest and the rigidity of the instrument that provides haptic feedback to the surgeon.

Preferably, the cutting instrument according to the invention has vertical reference recesses which can be detected by ultrasounds to identify a position of the instrument with respect to the surrounding tissues.

Preferably, the gripping portion comprises two portions facing each other with a left covering half shell and a right covering half shell coupled to each other to enclose both the end portion of the tie rod and the auxiliary element.

The cutting instrument according to the invention can have vertical reference recesses which can be detected by ultrasounds to identify a position of the instrument with respect to the surrounding tissues.

Further features and advantages will become more apparent from the following detailed description of preferred, but not exclusive, embodiments of the surgical cutting instrument according to the present invention, with reference to the enclosed figures given by way of non-limiting example.

### BRIEF DESCRIPTION OF THE DRAWINGS

In these drawings:

FIG. 1 shows a lateral view of a cutting instrument according to a first embodiment according to the invention;

FIG. 2 shows a sectional view of the cutting instrument of FIG. 1;

FIG. 3 shows an enlarged view of detail A in FIG. 2;

FIG. 4 shows an enlarged view of detail B in FIG. 2;

FIG. 5 shows a lateral view of the cutting instrument of FIG. 1 with the blade element in active position;

FIG. 6 shows a lateral view of a cutting instrument according to a second embodiment according to the invention;

FIG. 7 shows a sectional view of the cutting instrument of FIG. 6;

FIG. 8 shows an enlarged view of detail C in FIG. 6;

FIG. 9 shows an enlarged view of detail D in FIG. 7;

FIG. 10 shows an enlarged view of detail E in FIG. 7;

FIG. 11 shows the detail of FIG. 10 with the blade element in a partially extracted position

FIG. 12 shows a sectional view of the cutting instrument of FIG. 6 with the blade element in active position;

FIG. 13 shows an enlarged view of detail F in FIG. 12.

### DETAILED DESCRIPTION OF THE INVENTION

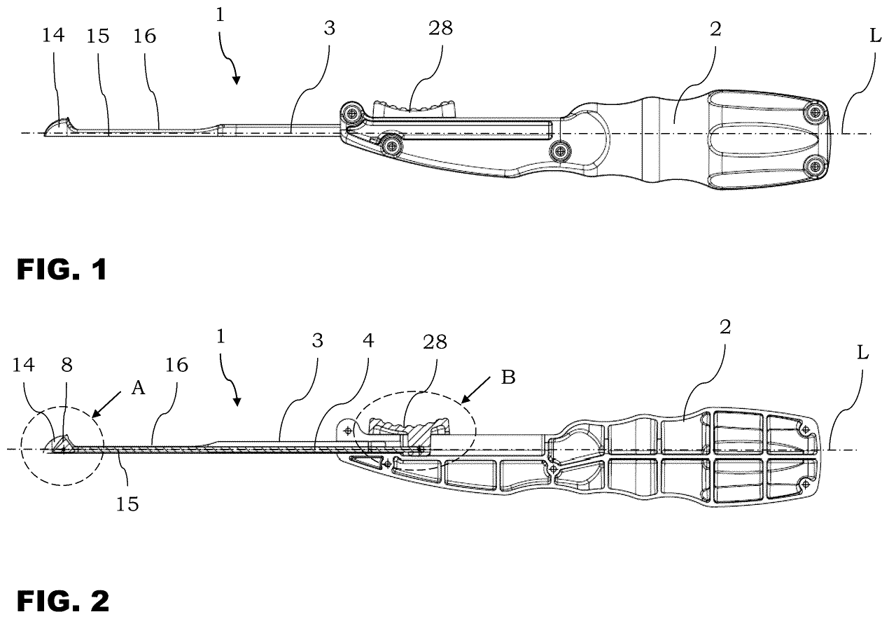

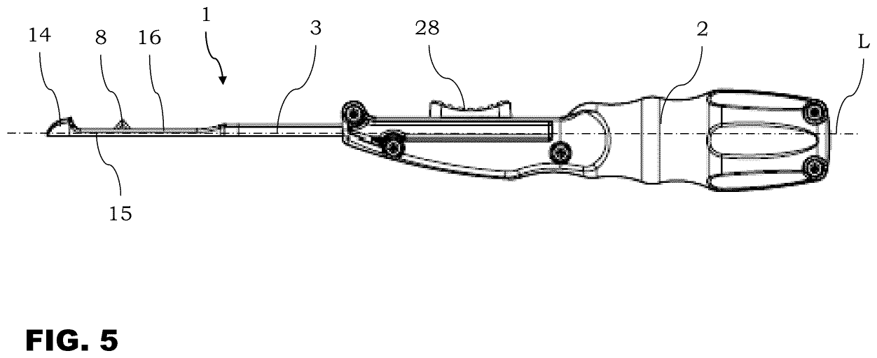

With reference to the enclosed FIGS. 1-5, reference number 1 globally and schematically indicates a first embodiment of a surgical cutting instrument made according to the present invention.

Specifically, the instrument 1 comprises a handle or gripping element 2. In the present embodiment this gripping element 2 is represented by a handle that can be grasped with just one hand by a surgeon in order to use the instrument 1.

A hollow stem 3, protruding from said gripping element 2 along a longitudinal axis L, is mounted on said gripping element 2.

In the present embodiment the gripping element 2 comprises a left gripping half shell and a right gripping half shell coupled to each other so as to enclose an end portion of the hollow stem 3.

Specifically, in said embodiment the assembly and constraint between left gripping half shell and right gripping half shell is made by means of screws but it is not limited as it could be made by means of ultrasonic welding, glue etc.

The left gripping half shell and the right gripping half shell are shaped so as to keep the hollow stem 3 in a projecting position along a longitudinal axis L.

In the hollow stem 3 a tie rod 4 is inserted or fitted through a proximal end 5 thereof. The rod 4 crosses the stem 3 until it penetrates into the gripping element 2 so that the proximal end 5 thereof is coupled to a sliding control element 28 housed in the gripping element 2, as visible in FIG. 4.

Opposite said proximal end 5, the tie rod 4 provides for a coupling portion 6 at a distal end 7.

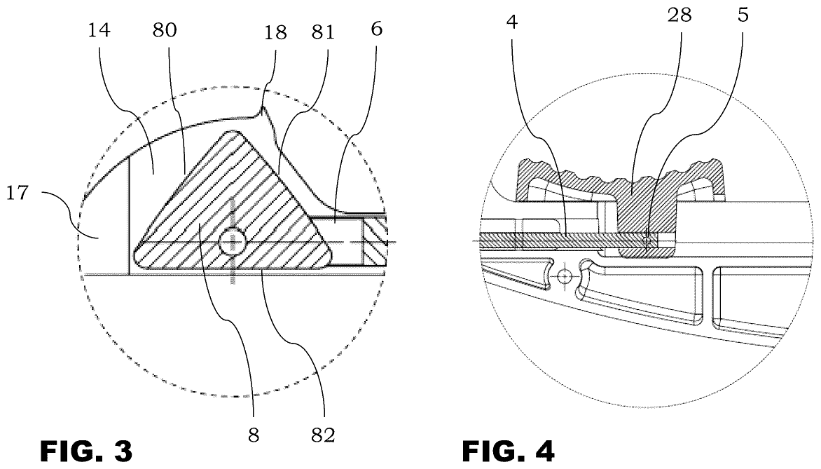

A blade element 8, which is triangle-shaped, is slidingly mounted within the hollow stem 3, and can be pulled by the tie rod 4 along the longitudinal axis L. More particularly, as more visible in FIG. 3, the distal end 6 of the tie rod 4 is coupled to the blade element 8.

The blade element 8 is triangular and comprises a first cutting edge 80, a second cutting edge 81 and a flat edge 82. The first and second cutting edges 80, 81 are oriented in two opposite directions of the longitudinal axis L, while the flat edge is a base planar element sliding along a bottom wall of the hollow stem 3. The blade element 8 does not rotate during its translation along the longitudinal axis L.

A distal segment of the hollow stem 3 comprises a distal covering tip 14, which is shaped to fully cover and contain the blade element 8; and an open portion 15, which is proximal with respect to the covering tip 14 and has an upper slit 16.

When the instrument is in its inactive position, meant for insertion within the patient and subsequent extraction, the blade element 8 rests in its housing within the covering tip, and therefore it is not exposed.

When the instrument is placed within the palmar arch in the patient body, the surgeon moves the actuating element 28, which in turn pulls the blade element 8 via the tie rod 4, from its distal inactive position to a proximal active position, wherein the blade element protrudes from the upper slit 16. The blade element 28 thereby slides along the open portion of the hollow stem and resects the ligament.

As visible in FIG. 3, the covering tip 14 comprises a nose 17 designed to facilitate the insertion of the instrument and a crest 18 that acts as a freer for the detachment of soft tissue.

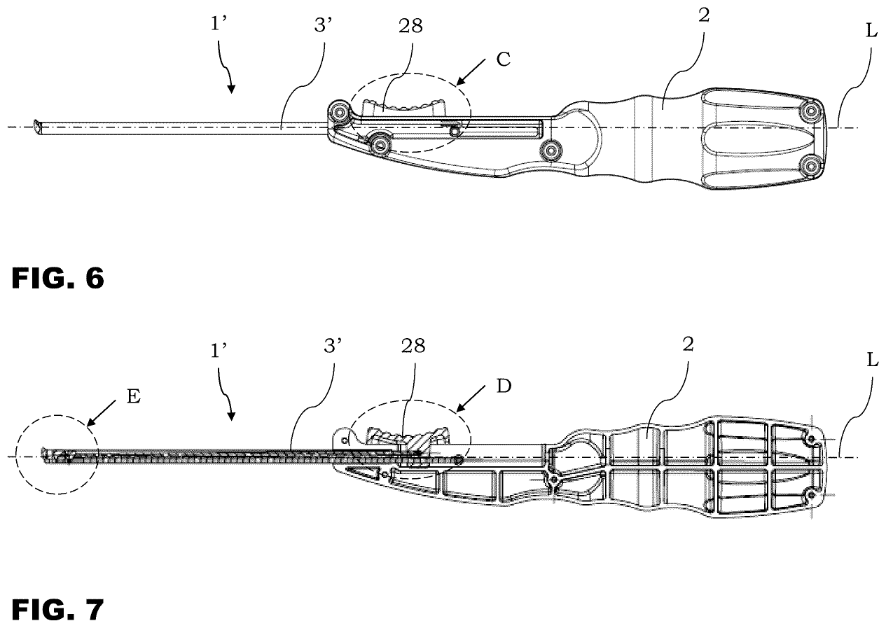

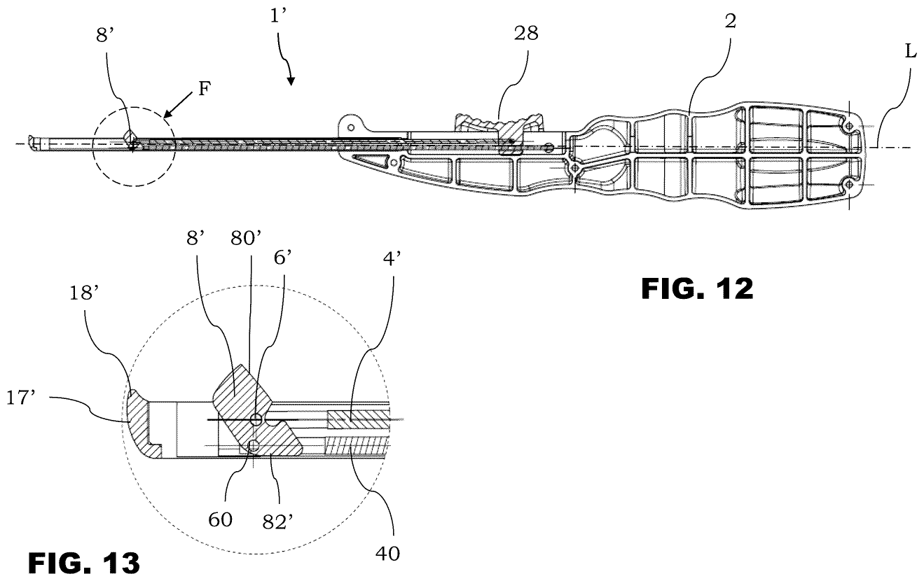

FIGS. 6-13 refer to a second alternative embodiment of a surgical cutting instrument 1′ according to the present embodiment.

The substantial differences compared to the first embodiment lie in the pulling device and in the shape of the hollow stem 3′.

The gripping element 2 and the actuating element 28 are identical with respect to the first embodiment, and therefore they are indicated with the same reference numbers.

In the second embodiment, the hollow stem 3′ has a constant width and does not comprise an enlarged tip for housing the blade element. However, this is merely a possible design and should be interpreted as a limitation. In an alternative design, there can be a distal widening of the stem or shaft in order to maintain the nerve and artery spaced apart.

In the illustrated embodiment, the blade element 8′ is substantially C-shaped, with only one straight cutting edge 80′ and a contiguous flat edge 82′ which is inclined at about 60° with respect to the cutting edge 80′. However, this is merely a possible design and should be interpreted as a limitation. An alternative design could employ a blade element with two cutting edges, which could be similar to the one discussed in connection with the first embodiment of the invention.

The blade element 8′ is free to translate along the longitudinal axis L and to rotate between an inactive position, in which it is parallel to the hollow stem 3′ and completely contained therewithin, and an active position, in which it is inclined at an angle of roughly 60° with respect to the hollow stem 3′ and sticks out of the hollow stem 3′ through an upper slit 16 thereof.

The surgical cutting instrument 1′ comprises a tie rod 4′, with a proximal end 5′ coupled to the actuating element 28 and a distal end which is hinged to a first hinging point of the blade element 8′.

An auxiliary element 40, which in the present embodiment is defined by a rod substantially parallel to the tie rod 4′, has a distal end 60 hinged to a second hinging point of the blade element 8′. This rod element will be hereinafter identified as the auxiliary rod 40.

As will be described in the following, a retaining device 54 is provided which selectively retains the auxiliary rod 40 in an initial position and releases it after a threshold pulling force is exerted thereon.

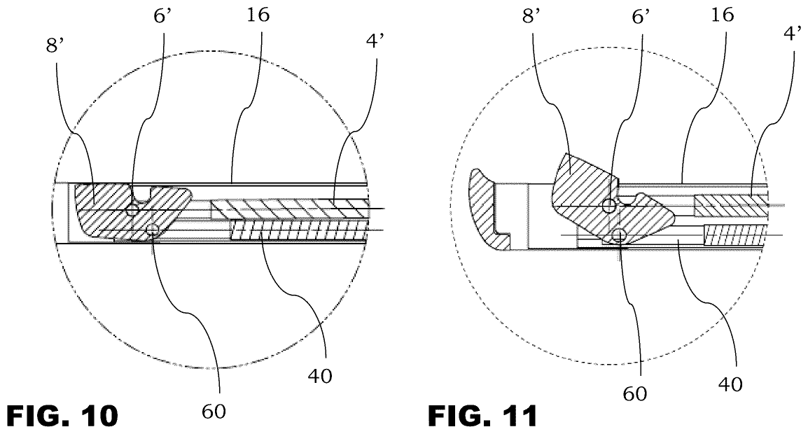

As visible in FIG. 10, the first hinging point 6′ and the second hinging point 60 are spaced apart both along the longitudinal direction L and a transversal direction when the blade element 8′ rests in its inactive position. When the tie rod 4′ is pulled in distal-to-proximal direction and the auxiliary rod 4 is retained, the blade element 8′ rotates about its second hinging point 60 and progressively raises in its active position, as best seen in FIGS. 11 and 13. In the active position, the two hinging points 6′, 60 are substantially aligned along the transversal direction. The tie rod 4′, which converges with the auxiliary rod 40 toward the blade element 8′ in the inactive position, progressively moves into a parallel relationship with the auxiliary rod 40.

Once the blade element 8′ is brought into its active position, its flat edge 82′ abuts against a bottom wall of the hollow stem 3′ and the pulling action exerted through the actuating element 28 is transmitted to the auxiliary rod 40, up to reaching the threshold force that disconnects the retaining device 54. Thereafter, the tie rod 4′ and auxiliary rod 40 move together in distal-to-proximal direction pulling the extracted blade element 8′ along the hollow stem 3′.

An opposite action on the actuating element 28 moves back the blade element 28 toward the tip of the instrument 1′ wherein it rotates back in its inactive position.

In other words, when the surgeon pulls the actuating element 28 the tie rod 4′ is pulled while the auxiliary rod 40 is kept in position by the retaining device 54. Therefore, the blade element 8′ rotates around its lower hinge, i.e. the second hinging point 54. Once the rotation is completed and the blade element 8′ extracted from its housing, if the surgeon keeps pulling the actuating element 28 he/she overcomes the threshold force induces a translation of the blade element 8′ by pulling both rods 4′, 40 at the same time. Once the surgeon has performed the resection of the transverse ligament, he/she pushes the blade element 8′ back toward the tip to retract the blade before extracting the instrument from the incision.

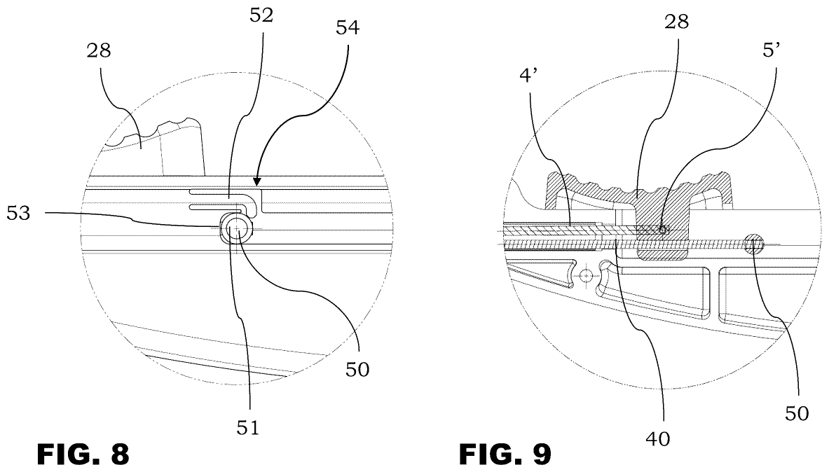

The above-mentioned retaining device 54, best visible in FIG. 8, is placed at the gripping element 2, proximal to the actuating element 28.

The retaining device 54 comprises a compliant retaining element in the form of an elastic tooth 52 which locks into place a pin 51 connected to a proximal end of the auxiliary rod 40. The elastic tooth 52 prevents motion in a distal-to-proximal direction of the pin 51. When a threshold force is reached, the elastic tooth 52 deflects upwards freeing the pin 51 and letting the auxiliary rod 40 move toward the proximal end of the gripping element 2.

The retaining device 54 further comprises an abutment 53 preventing the translation of the auxiliary element 40 in the proximal-to-distal direction out of the initial position.

As visible in FIG. 13, the surgical cutting instrument 1′ according to the second embodiment further comprises a tip 17′ coupled at the end of said hollow stem 3′ and comprising a protuberance 18′ for the detachment of the soft tissues, said protuberance 18′ being further adapted to provide a spatial positioning feedback of said surgical cutting instrument 1′ in working condition. However, in the second embodiment the tip is defined outside of the blade element 8′ pathway and it is not designed to house the blade element 8′.

In both embodiments described above, the blade element 8′ in its inactive position is completely hidden within the hollow stem 3, 3′. In this way, the surgeon can insert the instrument 1, 1′ into the palmar arch in complete safety without the risk of cutting unwanted tissue. The surgeon, indeed, operates in confined spaces and close to the median nerve and the ulnar artery. Therefore, it is absolutely essential that no damage is done to these tissues and nerve endings.

When the instrument is correctly positioned inside the carpal canal, by operating the actuating element 28 the movement is transmitted to the blade element 8, 8′ which moves into the active, extracted position. Them the blade element 8, 8′ slides horizontally in order to resect the transverse carpal ligament.

Afterwards, by reversing the sliding movement, the blade element 8, 8′ returns into its unactive, hidden position, allowing the surgeon to extract the instrument 1, 1′ from the carpal canal in complete safety.

Therefore, it is possible for a surgeon to operate in total safety even in the presence of multiple close-up tissues while maintaining a full haptic perception of the tissues touched.

The instrument of the present invention is simple to be made from a production point of view and therefore suitable for large-scale production and distribution.

In fact, the present invention allows speed and precision both in the production phase and in the assembly phase of the surgical cutting instrument, ensuring perfect covering of the blade element and the correct position thereof both in the active phase and in the inactive phase.

Also, the instrument according to the invention can be made with materials suitable for surgical instruments and capable of being sterilized and reused according to industry standards.

Advantageously, the present solution is optimal as regards the intervention at the carpal tunnel.

Advantageously, the present solution allows detaching and removing the soft tissues prior to carrying out the cutting of the ligament thanks to the freer that is designed on top of the protuberance, thus increasing the efficiency of the surgical procedure as there is no need to use a dedicated instrument as freer before entering with the cutting instrument, this will be done in one step with the same instrument. This will also increase the safety of the intervention allowing the surgeon to know the position of the inserted instrument thanks to the combination of the crest and of the rigidity of the instrument that provides haptic feedback to the surgeon.

Advantageously, in the first embodiment the wider body shape of the distal tip frees a space between the ulnar nerve and the artery so that the surgeon is sure that an accidental cut thereof is prevented.

Advantageously, this solution is practical for the surgeon and makes the overall cutting instrument compact.

Advantageously, the present solution is optimal as regards the intervention at the carpal tunnel.

The person skilled in the art will understand that the embodiments presented may be subjected to further changes and variations, according to specific and contingent needs, all included within the scope of protection of the invention as defined by the following claims.

Indeed, nothing prevents from providing variations in the conformation of components such as the blade element or the hollow stem according to different needs, while remaining within the scope of protection defined by said claims.

1. A surgical cutting instrument for minimally invasive surgery, comprising:
a gripping element;
an actuating element operatable by a surgeon at said gripping element;
a hollow stem mounted as an extension of said gripping element along a longitudinal axis (L) of the instrument;
a blade element slidable along the longitudinal axis (L) of the stem; and
a tie rod housed in the hollow stem and having a proximal end coupled to the actuating element and a distal end coupled to the blade element, so that a motion of the actuating element translates the blade element along the longitudinal axis (x) of the hollow stem;
wherein said hollow stem comprises: a distal covering tip fully covering the blade element; and an open portion proximal to said covering tip, transversally narrower than the blade element and comprising an upper slit;
wherein the motion of the actuating element moves the blade element from a distal inactive position, wherein the blade element is hidden within the covering tip, to a proximal active position, wherein the blade element protrudes from the upper slit; and vice versa.

2. The surgical cutting instrument according to claim 1, wherein the covering tip comprises a nose designed to facilitate the insertion of the instrument and a crest that acts as a freer for the detachment of soft tissue.

3. The surgical cutting instrument according to claim 1, wherein said blade element comprises a first cutting edge and a second cutting edge, the first and second cutting edges being oriented in two opposite directions of the longitudinal axis (L).

4. The surgical cutting instrument according to claim 1, wherein the actuating element is a slider translatable along the longitudinal axis (L) of the stem.

5. The surgical cutting instrument according to claim 3, wherein said blade element comprises a flat edge for sliding against a bottom wall of the hollow stem.

6. The surgical cutting instrument according to claim 3, wherein said blade element is triangular in shape.

7. The surgical cutting instrument according to claim 4, wherein said blade element is triangular in shape.

8. The surgical cutting instrument for minimally invasive surgery comprising:
a gripping element;
an actuating element operatable by a surgeon at said gripping element;
a hollow stem mounted as an extension of said gripping element along a longitudinal axis (L) of the instrument and comprising an upper slit;
a blade element slidable along the longitudinal axis (L) of the stem;
a tie rod extending along the longitudinal axis (L), housed in the hollow stem and having a proximal end coupled to the actuating element and a distal end rotatably coupled at a first hinging point of the blade element, so that a motion of the actuating element sets the blade element in motion;
an auxiliary element guided along the longitudinal axis (L) within the hollow stem and rotatably coupled at a second hinging point of the blade element, the second hinging point being spaced apart from the first hinging point; and
a retaining device that prevents translation from an initial position of the auxiliary element along the hollow stem in a distal-to-proximal direction until an unlocking pulling force is transmitted to the auxiliary element, and that locks the auxiliary rod in the initial position when pulled in a proximal-to-distal direction;
wherein the relative motion between the tie rod and the auxiliary element achieved when the tie rod is displaced by the actuating element and the auxiliary element is locked by the retaining device imparts a rotating motion to the blade element from an inactive position, wherein the blade element is hidden within the hollow stem, to an active position, wherein the blade element protrudes from the upper slit; and vice versa.

9. The surgical cutting instrument according to claim 8, wherein said auxiliary element is a rod having a proximal end selectively retained by the retaining device and a distal end rotatably coupled at the second hinging point of the blade element.

10. The surgical cutting instrument according to claim 8, wherein said retaining device comprises a compliant retaining element selectively preventing the translation of the auxiliary element in the distal-to-proximal direction.

11. The surgical cutting instrument according to claim 8, wherein said retaining device comprises an abutment preventing the translation of the auxiliary element in the proximal-to-distal direction.

12. The surgical cutting instrument according to claim 8, further comprising a tip coupled at the end of said hollow stem and comprising a protuberance for the detachment of the soft tissues, said protuberance being further adapted to provide a spatial positioning feedback of said surgical cutting instrument in working condition.

13. The surgical cutting instrument according to claim 8, further comprising a tip coupled at the end of said hollow stem and comprising a protuberance for the detachment of the soft tissues, said protuberance being further adapted to provide a spatial positioning feedback of said surgical cutting instrument in working condition.

14. The surgical cutting instrument according to claim 8, wherein said blade element is C-shaped.

15. The surgical cutting instrument according to claim 8, wherein the actuating element is a slider, translatable along the longitudinal axis (L) of the stem.

16. The surgical cutting instrument according to claim 9, wherein said retaining device comprises a compliant retaining element selectively preventing the translation of the auxiliary element in the distal-to-proximal direction.

17. The surgical cutting instrument according to claim 9, wherein said retaining device comprises an abutment preventing the translation of the auxiliary element in the proximal-to-distal direction.

18. The surgical cutting instrument according to claim 9, further comprising a tip coupled at the end of said hollow stem and comprising a protuberance for the detachment of the soft tissues, said protuberance being further adapted to provide a spatial positioning feedback of said surgical cutting instrument in working condition.

19. The surgical cutting instrument according to claim 9, further comprising a tip coupled at the end of said hollow stem and comprising a protuberance for the detachment of the soft tissues, said protuberance being further adapted to provide a spatial positioning feedback of said surgical cutting instrument in working condition.

20. The surgical cutting instrument according to claim 9, wherein said blade element is C-shaped.

21. The surgical cutting instrument according to claim 9, wherein the actuating element is a slider translatable along the longitudinal axis (L) of the stem.

22. The surgical cutting instrument according to claim 9, wherein said retaining device is placed on the gripping element, optionally in the vicinity of the actuating element.

23. The surgical cutting instrument according to claim 10, wherein said blade element is C-shaped.

24. The surgical cutting instrument according to claim 10, wherein said retaining device comprises a compliant retaining element selectively preventing the translation of the auxiliary element in the distal-to-proximal direction.

25. The surgical cutting instrument according to claim 10, wherein said retaining device comprises an abutment preventing the translation of the auxiliary element in the proximal-to-distal direction.

26. The surgical cutting instrument according to claim 15, wherein said slider is partially housed and guided within the gripping element.

27. The surgical cutting instrument according to claim 26, wherein said retaining device comprises a compliant retaining element selectively preventing the translation of the auxiliary element in the distal-to-proximal direction.

28. The surgical cutting instrument according to claim 27, wherein said compliant retaining element is an elastic tooth acting on a pin connected to the auxiliary element.
